(19) United States
(12) Patent Application Publication
(10) Pub. No.: US 2024/0180914 A1
(43) Pub. Date: Jun. 6, 2024

(54) ORAL GEL COMPOSITION AND USE THEREOF

(71) Applicant: DR. KURT WOLFF GMBH & CO. KG, Bielefeld (DE)

(72) Inventors: Erik SCHULZE ZUR WIESCHE, Bielefeld (DE); Joachim ENAX, Bielefeld (DE); Frederic MEYER, Bielefeld (DE)

(73) Assignee: DR. KURT WOLFF GMBH & CO. KG, Bielefeld (DE)

(21) Appl. No.: 18/285,021

(22) PCT Filed: Apr. 28, 2022
(86) PCT No.: PCT/DE2022/100316

§ 371 Date: Sep. 29, 2023

(30) Foreign Application Priority Data
May 3, 2021 (DE) 10 2021 111 387.3

(51) Int. Cl. A61K 31/522; A61K 9/00; A61K 9/06; A61K 47/38; A61P 1/02

(52) U.S. Cl. CPC A61K 31/522; A61K 9/0063; A61K 9/06; A61K 47/38; A61P 1/02

(57) ABSTRACT

An oral gel composition to be applied to the gums and to remain thereon for at least 3 minutes, is used for oral care, and in particular for effective treatment of (i.e. reducing and preventing) periodontitis/gingivitis and gum recession. The oral gel composition includes at least 0.00001 and less than 0.5% by weight caffeine and a gelling agent. The oral gel composition allows cell metabolism to be activated and thus allows effective treatment and/or prevention of periodontitis/gingivitis and gum recession.

The present invention relates to an oral gel composition to be applied to the gums and to remain thereon for at least 3 minutes, and to the use thereof.

Simply because of the increasingly high-carbohydrate diet worldwide, dental and gum care is becoming more and more important. Therefore, in addition to aesthetic aspects, particular emphasis is increasingly being placed on preventive care, wherein the focus is primarily on reducing or even avoiding plaque, caries, and/or halitosis (bad breath) as well as healthy gums.

One of the features of the gums is that they surround the teeth cervically. This means that the gums surround the neck of the tooth, sealing the entry point of the tooth into the jawbone of the oral cavity. The gums therefore serve, among other things, to protect the tooth and hold it in place.

The different parts of a natural tooth are the crown, neck and root, wherein these are made up of several layers. Of these layers, you normally only see the outer enamel (enamelum), which surrounds the dentin and other layers. For example, in order to be able to bite or grind food without damaging the teeth, the enamel is very hard. It consists of about 97% by weight of hydroxyapatite, which has the molecular formula Ca₅(PO₄)₃(OH). The dentin is also considered to be hard tooth tissue and also consists of about two-thirds hydroxyapatite. In addition to hydroxyapatite, dentin also contains proteins and water and is therefore not as hard as enamel.

Common dental and gum diseases such as caries and periodontitis/gingivitis can be based on the formation of bacterial microfilms and/or be due to bacterial inflammation. Furthermore, the problem of receding gums is widespread and occurs very frequently, in particular with increasing age.

DE 298 06 937 U1 describes a toothpaste to which one or more stimulating substances are added, which may be, for example, caffeine or nicotine.

DE 201 19 966 U1 also describes a toothpaste enriched with invigorating substances, wherein caffeine or other stimulating substances are included in the toothpaste formulation.

A caffeine-containing toothpaste is also known from JP 2010/275261 A1.

The publication “Neurophysiological effect of flavor and caffeine added to toothpaste”, Sangyo Eiseigaku Zasshi, vol. 52, issue 4, pages 172-181, 2010:1478424 CAN156:675063 also discloses enriching toothpaste with caffeine, among other things, to reduce fatigue.

WO 2021/013283 A1 discloses compositions for oral hygiene means, in particular toothpastes, which contain caffeine and lysine-containing bitter blockers. Since oral hygiene products are usually not swallowed but spat out, the caffeine must be present in a sufficiently high concentration for it to be effective. The bitter blockers serve to achieve a pleasant taste experience, even at these very high caffeine concentrations.

Finally, a mouthwash containing caffeine is also offered by Johnson & Johnson under the name “LISTERINE GREEN TEA”.

However, the dwell time of toothpaste or mouth rinse/mouthwash in the oral cavity is limited to about 30 seconds to a maximum of 3 minutes, so that only little caffeine can interact with the gums during the duration of dental care. Afterwards, these are usually spat out and also rinsed with water. This causes the effective concentration of caffeine in the oral cavity to decrease very quickly. Such oral hygiene products serve to care for, moisten and kill bacteria, but have no long-term positive effect on the cells of the gums.

From DE 10 2019 119 589 A1 an oral hygiene agent containing caffeine and a bitter blocker is known, wherein the bitter blocker consists of 8% L-lysine hydrochloride and 92% maltodextrin and the concentration of caffeine per portion of the oral hygiene agent is between 5 and 200 mg.

US 2013/0129641 A1 describes compositions for caries prevention with a calcium source and a phosphate source, which has a pH value of 6.0 to 8.5. These compositions contain theobromine.

The present invention is based on the object of providing a product for oral care, and in particular for effective treatment of (i.e. reducing and preventing) periodontitis/gingivitis and gum recession.

Furthermore, the use of the oral gel should not significantly disturb the ecological balance in the oral cavity and/or risk tooth discolouration or taste disturbance.

These objects are achieved according to the invention in that the oral gel composition contains
(a) at least 0.00001 and less than 0.5% by weight caffeine and
(b) a gelling agent.

Caffeine is a methylxanthine, which surprisingly has been shown to be superior to other methylxanthines, such as theobromine or theophylline, in its effect on gum regeneration. In particular, the subjective perception of use of caffeine-based tooth gel, in terms of taste, effect on the gums, reduction of bleeding gums, was found to be significantly better than that of theobromine-based or theophylline-based tooth gel in an application study with 50 participants. Moreover, in this application study, caffeine concentrations of less than 0.5% by weight were surprisingly better evaluated than higher caffeine concentrations (of more than 0.5%).

Unlike a dental gel, the oral gel according to the invention is not applied to the teeth but to the gums. It could therefore also be called gum gel, as its primary purpose is to care for and treat the gums.

In contrast to prior art oral care products containing caffeine, the dwell time of the oral gel according to the invention in the oral cavity is many times longer, as it is not rinsed out. This results in a longer interaction of the caffeine with the gums. Furthermore, the oral gel according to the invention is not spat out but slowly dissolved via saliva, resulting in more advantageous release kinetics than with prior art oral care products. Therefore, this is not a leave-on product. In addition, the oral gel according to the invention does not contain any cleaning agents, which enables a gentle treatment especially for acutely irritated gums.

A smaller amount of caffeine than in the oral care products that only act for a short time is therefore more beneficial. Surprisingly, it has been found within the scope of the invention that it is advantageous if smaller amounts of caffeine (<0.5% by weight) are available over a longer period of time, because this results in a “deposit effect”. The disadvantage of higher caffeine concentrations is that cell-damaging effects can occur.

In the oral gel according to the invention, the dwell time on the gum is between 3 minutes and 72 hours, preferably between 5 minutes and 48 hours, in particular between 10 minutes and 24 hours. Such long dwell times are not achieved with any of the known oral care products.

It has unexpectedly been determined that the oral gel composition according to the invention allows cell metabolism to be activated and thus allows effective treatment and/or prevention of periodontitis/gingivitis and gum recession. In the context of the present invention, the oral gel composition according to the invention may comprise both cosmetic and medical care products.

Furthermore, it was found that the use of the oral gel composition according to the invention supports the adhesion of the caffeine directly to the gum or gumline, so that the caffeine is in direct contact therewith for a longer period of time, thus achieving a high absorption rate.

Here, the caffeine is dosed differently depending on the user group. A daily dose of 0.3 mg caffeine/kg body weight (EFSA NDA Panel (EFSA Panel on Dietetic Products, Nutrition and Allergies, 2015, Scientific opinion on the safety of caffeine, EFSA Journal 2015; 13 (5); 4102, 120 pp. Doi:10.2903/j.efsa.2015.4102) should not be exceeded, which can be absorbed both via a single application and via several applications over the course of a day (wherein 1 to 3 applications per day are preferred).

Preferably, the oral gel composition contains caffeine in an amount of from 0.0001 to 0.25% by weight, preferably from 0.001 to 0.1% by weight and particularly preferably from 0.01 to 0.05% by weight, in particular 0.025% by weight, each in relation to the total weight of the oral gel composition.

According to the invention, the composition is an oral gel, i.e. a gel to be applied in the mouth (region). The gel is applied to the gums. This application can be done using the fingers or also with a suitable device such as a cotton swab, a brush or a toothbrush. Alternatively, the gel can also be filled into a bite guard, which can then be worn over a longer period of time, such as overnight. By applying the gel to the gums as described above, long-term contact between the gel and the gums is achieved, thus ensuring intensive use.

In the context of the present invention, a gel is understood to be a disperse system consisting of at least two components. The solid component is called a gelling agent and can form a three-dimensional network in the process. The liquid component is stored in the interstices of the solid component and is thus immobilised. The two components thus interpenetrate.

In a preferred embodiment, the liquid component is water.

Consequently, the oral gel composition according to the invention comprises a gelling agent as essential ingredient (b).

Gelling agents can be synthetic or natural polymer materials. The natural polymer materials are preferably plant-based polymer materials.

In a preferred embodiment, the gelling agent comprises methyl cellulose, ethyl cellulose, hydroxyethyl cellulose, hydroxypropyl cellulose, hydroxypropyl methyl cellulose, carboxymethyl cellulose, sodium carboxymethyl cellulose, agaraose, agar, scelrotium gum, xanthan gum, guar gum, carrageenan, polyvinylpyrrolidone and mixtures thereof, particularly preferably hydroxyethyl cellulose, hydroxypropyl cellulose, hydroxypropyl methyl cellulose, carboxymethyl cellulose, agaraose, agar, scelrotium gum, guar gum, carrageenan, polyvinylpyrrolidone and mixtures thereof.

Very particularly, the gelling agent consists of a natural raw material, preferably selected from the group of celluloses and xanthans.

Suitable celluloses include hydroxyethyl cellulose, hydroxypropyl cellulose, hydroxypropyl methyl cellulose, carboxymethyl cellulose, and most preferably hydroxyethyl cellulose.

In a further preferred embodiment, the oral gel composition according to the invention contains gelling agent in an amount of from 0.01 to 4.0% by weight, particularly preferably from 1.5 to 3.0% by weight and in particular approximately 2.0% by weight, each in relation to the total weight of the oral gel composition.

In a preferred embodiment, the oral gel composition further comprises (c) one or more pH regulators and/or (d) one or more pharmaceutical or cosmetic ingredients.

pH regulators (c) are substances used to adjust a composition to a specific pH value or range of pH values.

Examples of pH regulators include acetic acid, acetates, lactic acid, lactates, malic acid, malates, fumaric acid, citric acid, citrates, tartaric acid, tartrates, orthophosphates, di-, tri- and polyphosphates, hydrochloric acid, chlorides, sulphuric acid, sulphates, hydroxides, oxides, adipic acid, adipates, gluconic acid, gluconates, phosphoric acid, calcium carbonate or a hydrate thereof. A preferred example of a pH regulator that can be added when a lower pH is desired is phosphoric acid (H₃PO₄).

The oral gel composition according to the invention preferably does not have an acidic pH of less than 5.5. If the composition is too acidic, there would be a risk of demineralisation of the tooth structure (erosion).

Preferably, the oral gel composition according to the invention has a pH value in the range from 5.0 to 9.0, more preferably a pH value in the range from 6.5 to 8.0, in particular a pH value of approximately 7.0. It has been found that the remineralisation of the tooth structure is favoured by a pH value in the above-mentioned range. Preferably, the pH regulator comprises (c) calcium carbonate or a hydrate thereof. Alternatively, the pH regulator comprises a mixture of (c) calcium carbonate (or a hydrate thereof) and sodium hydroxide.

In a further preferred embodiment, the oral gel composition according to the invention contains a pH regulator (c) in an amount of from 0.05 to 3.0% by weight, more preferably from 0.1 to 2.5% by weight, particularly preferably 0.2 to 1.5% by weight, each in relation to the total weight of the oral gel composition.

In a preferred embodiment, the oral composition according to the invention can contain one or more pharmaceutical or cosmetic ingredients. These pharmaceutical or cosmetic ingredients are described, for example in Toothpastes, Monographs in Oral Science, Vol. 23, 1st edition, 2013.

Preferably, the pharmaceutical or cosmetic ingredients comprise antimicrobial substances, flavouring agents and sugar alcohols.

Antimicrobial substances are substances that can kill microorganisms, such as bacteria, or greatly reduce their proliferation. In addition to antimicrobial substances with a non-specific defence against bacteria and fungi, there are also substances that are only effective against specific bacteria, for example. The use of antimicrobial substances can also combat bad breath, for example. Preferably, antimicrobial substances may be present in an amount of from 0.01 to 1.0% by weight, preferably from 0.05 to 0.5% by weight, in the oral gel composition according to the invention.

Examples of antimicrobial substances used in oral care are zinc compounds such as zinc chloride and zinc citrate, as well as chlorhexidine, triclosan, cetylpyridinium chloride and stannous chloride. Particularly preferably, the oral gel composition according to the invention does not contain chlorhexidine.

Flavouring agents can give the oral composition according to the invention the desired flavour. In addition, flavouring agents can stimulate saliva, wherein the moisture of the saliva can have a positive influence on the remineralisation of the tooth. An example of a saliva-stimulating flavouring agent is pellitorin, in particular trans-pellitorin. Other examples of flavouring agents are the commercially available flavourings “Aktiv Fresh P0128844” and “Citromint P0125337” as well as menthol.

In a preferred embodiment, the pharmaceutical or cosmetic ingredient (d) comprises sugar alcohols, in particular xylitol or sorbitol. Xylitol can minimise the number of caries bacteria and inhibit their growth. Furthermore, xylitol can stimulate the flow of saliva. The increased amount of saliva results in an increased amount of phosphate. This phosphate can react together with the calcium (ions) from the oral gel composition according to the invention to form hydroxyapatite. The oral gel composition according to the invention can contain xylitol in an amount of from 0.5 to 4% by weight, preferably from 0.7 to 2.5% by weight, in particular approximately 1.0% by weight in relation to the total weight of the oral gel composition. In addition to or instead of xylitol, the composition according to the invention can also contain sorbitol.

In a further preferred embodiment, the oral gel according to the invention comprises a suitable bitter blocker (e). This bitter blocker consists of between 0.0001 to 15% by weight, in one embodiment between 2 and 12% by weight, in a further embodiment between 3 and 10% by weight, in a further embodiment 1, 2, 3, 4, 5, 6, 7, 8 or 9% by weight of L-lysine, in a further embodiment in the same amounts of L-lysine hydrochloride, and a bitter blocker carrier. Here, as much bitter blocker carrier is added so that a total percentage value of 100% by weight is achieved for the bitter blocker itself, e.g. 85% by weight bitter blocker carrier with 5% by weight bitter blocker.

Alternatively, it is also possible that the oral gel according to the invention is free of bitter blockers, which is the case in particular at lower caffeine concentrations. Bitter blockers can negatively influence the overall taste sensation.

Conventional dental care compositions such as oral gels often contain fluoride compounds such as sodium fluoride, amine fluorides or stannous fluoride. However, in a particularly preferred embodiment of the present invention, the oral gel composition according to the invention contains precisely no fluoride compound and is thus substantially or even completely fluoride-free.

Furthermore, known dental care compositions such as GC's Tooth Mouse contain animal ingredients such as casein phosphopeptide components. In a particularly preferred embodiment, the oral gel composition according to the invention does not contain any animal components and is therefore vegan.

In a preferred embodiment, the oral gel composition according to the invention contains
at least 0.00001 and less than 0.5% by weight, preferably 0.0001 to 0.01% by weight, more preferably 0.0005 to 0.001, particularly preferably 0.005% by weight caffeine,
5 to 25% by weight, preferably 10 to 20% by weight, more preferably 12 to 18% by weight, in particular approximately 15% by weight Ca₅(PO₄)₃(OH),
0.5 to 5% by weight, preferably 1 to 4% by weight, more preferably 1.5 to 3% by weight, in particular approximately 2.0% by weight 0.15 to 0.5% by weight one or more calcium salts selected from calcium chloride, calcium bromide, calcium nitrate, calcium acetate, calcium gluconate, calcium lactate, calcium tartrate and hydrates and mixtures thereof
1 to 4% by weight, preferably 1.5 to 3% by weight, in particular approximately 2% by weight, gelling agent—0.05 to 3.0% by weight, preferably 0.1 to 2.5% by weight, in particular 0.2 to 1.5% by weight pH regulator
0.5 to 15% by weight, preferably 1 to 12% by weight one or more pharmaceutical or cosmetic ingredients, wherein the percentages by weight relate to the total weight of the oral gel composition. The pH value is in a range from pH 6.5 to 8.5.

Preferably, the oral gel composition is free of fluorides and/or tin salts and/or chlorhexidine and/or cetylpyridinium chloride and/or triclosan.

Tin salts such as stannous fluoride and stannous chloride cause discolouration of the teeth. Chlorhexidine also leads to discolouration of the teeth and impaired taste. Cetylpyridinium chloride can also cause discolouration of the teeth. With triclosan, there is a risk of resistance forming.

Furthermore, the present invention relates to the use of the oral gel composition according to the invention for the treatment and/or prevention of periodontitis/gingivitis or gum recession.

In this context, the dwell time of the oral gel composition on the gums is preferably between 3 minutes and 72 hours, preferably between 5 minutes and 48 hours, more preferably between 10 minutes and 24 hours.

In the following, the invention is explained by means of examples.

### EXAMPLES

Oral gel composition

### 1.1 Water Phase

A water phase containing the following components was prepared. Demineralised water 2247.5 g
1,2-hexanediol and 1,2-octanediol (SymDiol 68) 25.0 g
Hydroxyacetophenone (SymSave H) 25.0 g
Calcium lactate 100.0 g
Xylitol (Xylisorb 700) 50.0 g
Stevia (Reb A97) 10.0 g
   1.2 Gelling agent phase
A gelling agent containing the following components was prepared.
Sorbitol solution 70% 850.0 g
Glycerin 85% (vegetable Ph. Eur.) 750.0 g
Hydroxyethyl cellulose (Natrosol 250 H) 75.0 g
   1.3 Flavouring phase
A flavouring phase containing the following components was prepared.
Flavouring (Aktiv Fresh P0128844) 5.0 g
Flavouring (Citromint P0128337) 5.0 g
PEG-40 hydrogenated castor oil/propylene glycol/aqua (Eumulgin CO455) 75.0 g
   1.4 Caffeine phase including pH adjustment
A caffeine phase containing the following components was prepared.
Caffeine 0.1%
Caustic soda 45% 7.50 g

An oral gel was prepared by combining and mixing the above phases 1.1 to 1.4, wherein the composition of the oral gel contains the following ingredients:
Demineralised water 2247.5 g (44.95%)
1,2-hexanediol and 1,2-octanediol (SymDiol 68) 25.0 g (0.5%)
Hydroxyacetophenone (SymSave H) 25.0 g (0.5%)
Calcium lactate 100.0 g (2.0%)
Xylitol (Xylisorb 700) 50.0 g (1.0%)
Stevia (Reb A97) 10.0 g (0.2%)
Sorbitol solution 70% 850.0 g (17.0%)
Glycerin 85% (vegetable Ph. Eur.) 750.0 g (15.0%)
Hydroxyethyl cellulose (Natrosol 250 H) 75.0 g 0 g (1.5%)
Flavouring (Aktiv Fresh P0128844) 5.0 g (0.1%)
Flavouring (Citromint P0128337) 5.0 g (0.1%)
PEG-40 hydrogenated castor oil/propylene glycol/aqua (Eumulgin CO455) 75.0 g
(1.5%) calcium carbonate (Precafood 1) 25.0 g (0.5%)
Caffeine X g (X 0%)
Caustic soda 45% 7.50 g (0.15%)

The percentages given correspond to percentages by weight in relation to the total weight of the composition.

The oral gel is a white, homogeneous gel with a lemony, minty, sweet taste and has an average pH value of 7.7.

Study 1: Effect of caffeine on the biological functions of human epidermal HaCaT keratinocytes (viability study)

To investigate the effect of caffeine on the biological functions of human epidermal HaCaT keratinocytes, cell experiments were performed on human epidermal HaCaT keratinocytes, wherein the following studies were carried out:
1. P13-A biological assay 1 with two independent studies
2. P13-B biological assay 2 with two independent studies.

The initial cell count was 5000 cells/well in 100 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0001% caffeine
4. 0.0005% caffeine
5. 0.001% caffeine
6. 0.005% caffeine

The trial durations were 24, 48 and 72 hours.

The study took the form of a colourimetric MTT study.

The results are presented in

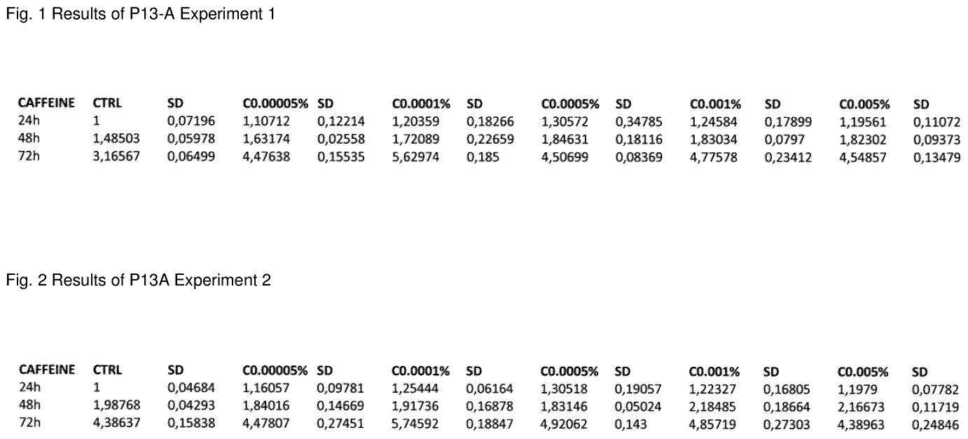

FIG. 1 Results of P13-A Experiment 1

FIG. 2 Results of P13-A Experiment 2

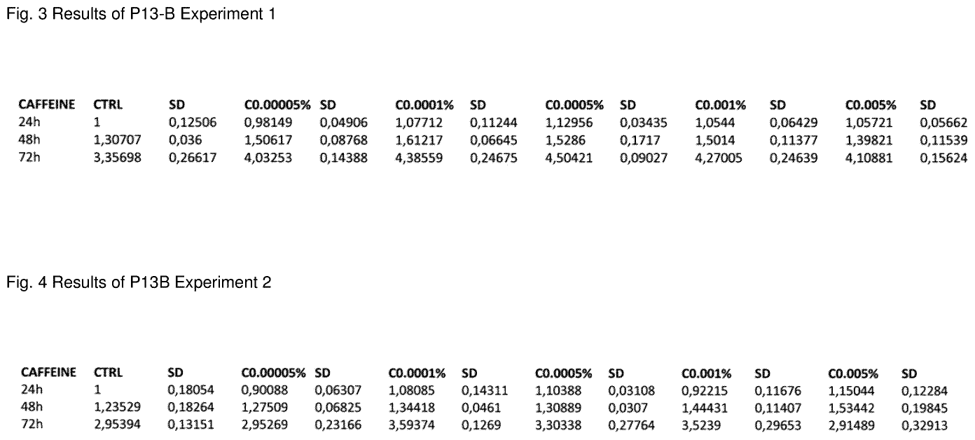

FIG. 3 Results of P13B Experiment 1

FIG. 4 Results of P13-B Experiment 2 and

The study found that none of the concentrations tested showed cytotoxicity even after 72 hours of testing and that certain concentrations of caffeine increased the number of viable cells.

Study 2: Effect of caffeine on the biological functions of human epidermal HaCaT keratinocytes (cell proliferation study)

To investigate the effect of caffeine on cell proliferation of human epidermal HaCaT keratinocytes, human HaCaT keratinocytes were subjected to a CyQUANT viability assay, wherein the following series of experiments were performed:
1. P22 Experiment 1 (biological assay 1)
2. P24 Experiment 2 (biological assay 2).

The initial cell count was 5000 cells/well in 1,500 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0005% caffeine
4. 0.005% caffeine

The trial durations were 24, 48 or 72 hours.

The study was carried out by fluorometric CyQUANT study, wherein the DNA content was measured, reflecting cell proliferation.

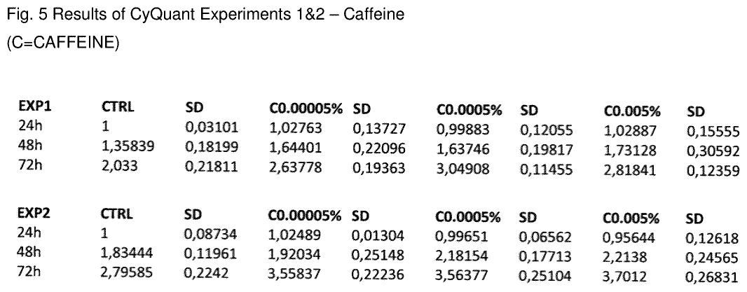

The results are presented in
FIG. 5 Results of CyQuant Experiments 1&2 Caffeine

The study shows that caffeine appears to promote cell proliferation; the best proliferation-enhancing effects were found after 72 hours.

Study 3: Effect of caffeine on the biological functions of human epidermal HaCaT keratinocytes (cell proliferation study)

To investigate the effect of caffeine on the biological functions of human epidermal HaCaT keratinocytes, human HaCaT keratinocytes were subjected to Q-PCR and ELISA, wherein the following series of experiments were performed:
1. For Q-PCR: Determination of VEGF, HGF and EGF mRNA expression in cell lysates
2. For ELISA: Determination of VEGF, HGF and EGF protein synthesis/secretion into the culture medium

The initial cell count was
1. for Q-PCR: 140,000 cells/well in 1500 μl culture medium
2. for ELISA: 140,000 cells/well in 1.500 μl culture medium.

The culture medium was HaCaT medium (DMEM with the addition of 10% FBS, 1% penicillin-streptomycin, 0.5% fungizone).

The treatment groups were as follows:
1. Control group (CTRL)
2. 0.00005% caffeine
3. 0.0005% caffeine
4. 0.005% caffeine

The trial duration for
1. Q-PCR was 24 hours (single treatment at time 0 h)
2. ELISA was 48 hours (double treatment at time 0 h and 24 h without changing the medium).

The study was carried out
1. for Q-PCR by determining VEGF mRNA expression in cell lysates
2. for ELISA by determining VEGF protein synthesis/secretion in the culture medium.

The results are presented in

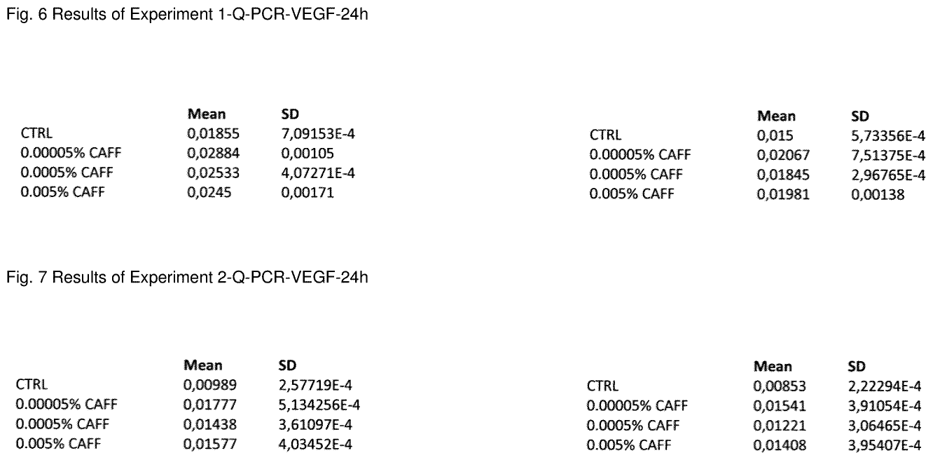

FIG. 6 Results of Experiment 1-Q-PCR-VEGF-24 h

FIG. 7 Results of Experiment 2-Q-PCR-VEGF-24 h

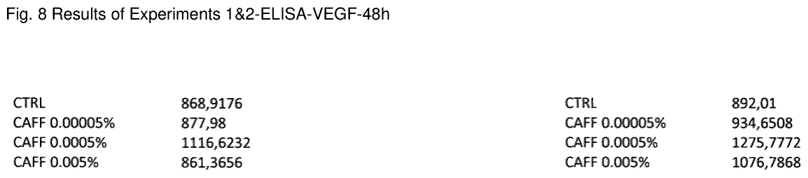

FIG. 8 Results of Experiments 1&2-ELISA-VEGF-48 h

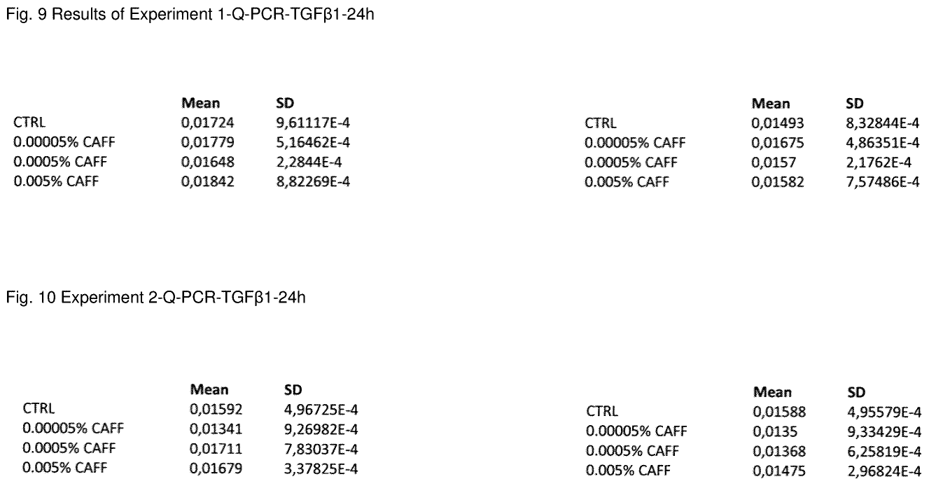

FIG. 9 Results of Experiment 1-Q-PCR-TGFβ1-24 h

FIG. 10 Results of Experiment 2-Q-PCR-TGFβ1-24 h

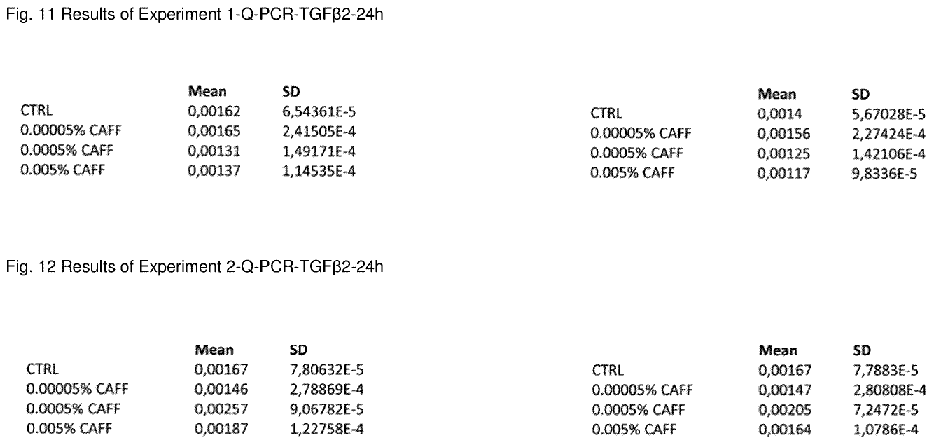

FIG. 11 Results of Experiment 1-Q-PCR-TGFβ2-24 h

FIG. 12 Results of Experiment 2-Q-PCR-TGFβ2-24 h

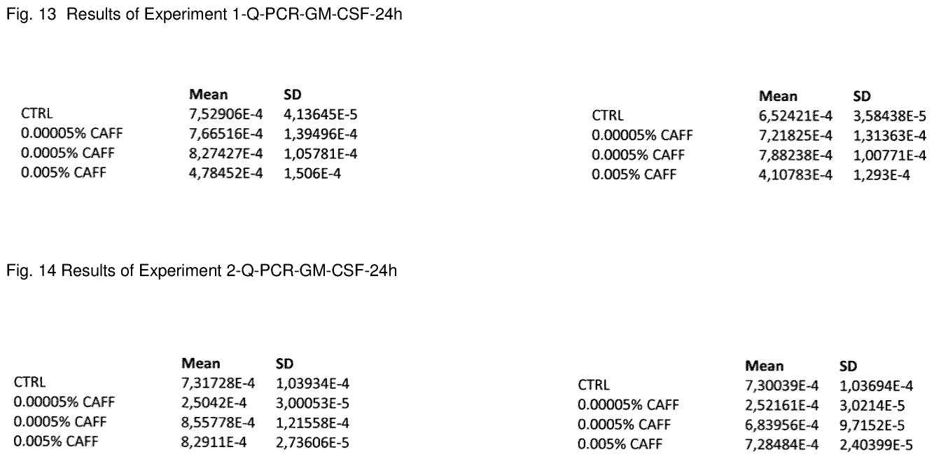

FIG. 13 Results of Experiment 1-Q-PCR-GM-CSF-24 h

FIG. 14 Results of Experiment 2-Q-PCR-GM-CSF-24 h

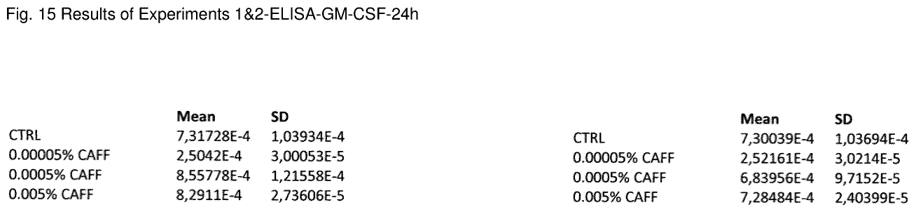

FIG. 15 Results of Experiments 1&2-ELISA-GM-CSF-48 h.

The study found the following:
Q-PCR (24 hours treatment time)—Experiments 1 and 2: All caffeine concentrations appear to increase mRNA expression of VEGF compared to the control group.
ELISA (48 hours treatment time)—Experiments 1 and 2: 0.0005% caffeine increases VEGF protein production/secretion compared to the control group in both experiments. In Experiment 2, 0.005% caffeine also showed an effect.

Taken together, this data suggests that certain concentrations of caffeine increase the synthesis and production/secretion of VEGF in cultured human epidermal HaCaT keratinocytes.

The expression of HGF-specific mRNA, HGF-specific protein, TNFα-specific mRNA, TNFα-specific protein and TGFβ1-specific protein was below the detection limit in both experiments.

The experiments regarding GM-CSF show the following:
Q-PCR-Experiment (24 h treatment time)—Experiments 1 and 2: All tested caffeine concentrations apparently increased the mRNA expression of VEGF compared to the control group.
ELISA (48 h treatment time)—Experiments 1 and 2: 0.0005% caffeine increased VEGF protein production/secretion compared to the control group in both experiments. In Experiment 2 0j.005% caffeine also showed an effect.

Taken together, this data strongly suggests that certain concentrations of caffeine increase the synthesis/production/secretion of VEGF in cultured human epidermal HaCaT keratinocytes.
Q-PCR-Experiment (24 h treatment time)—Experiments 1 and 2: None of the tested caffeine concentrations changed the mRNA expression of TGFβ1. However, in Experiment 2, certain caffeine concentrations apparently increase mRNA expression of TGFβ2.
ELISA (48 h treatment time)—Experiments 1 and 2: The level of TGFβ1-specific protein was below the detection limit in both experiments.

Taken together, this data suggests—despite the contradictory results in the two Q-PCR experiments—that caffeine most likely does not alter the synthesis/production/secretion of TGFβ2 in cultured human epidermal HaCaT keratinocytes.

The experiments regarding TGFβ1 showed the following:
Q-PCR-Experiment (24 h treatment time)—Experiments 1 and 2: None of the tested caffeine concentrations changed the mRNA expression of TGFβ1.
ELISA (48 h treatment time)—Experiments 1 and 2: The level of TGFβ1-specific protein was below the detection limit in both experiments.

Taken together, this data suggests that caffeine does not alter the synthesis/production/secretion of TGFβ1 in cultured human epidermal HaCaT keratinocytes.

1. An oral gel composition to be applied to the gums and to remain thereon for at least 3 minutes, comprising
(a) at least 0.00001 and less than 0.5% by weight caffeine and
(b) a gelling agent.

2. The oral gel composition according to claim 1, wherein the oral gel composition contains caffeine in an amount of from 0.0001 to 0.25% by weight, preferably from 0.001 to 0.1% by weight and particularly preferably from 0.01 to 0.05% by weight, in particular 0.025% by weight, each in relation to the total weight of the oral gel composition.

3. The oral gel composition according to claim 1, wherein the gelling agent is selected from the group containing cellulose and cellulose derivatives such as methyl cellulose, ethyl cellulose, hydroxyethyl cellulose, hydroxypropyl cellulose, hydroxypropyl methyl cellulose, carboxymethyl cellulose, sodium carboxymethyl cellulose, agaraose, agar, pectins, sclerotium gum, xanthan gum, guar gum, carrageenan, alginic acid, polyvinyl alcohol, polyvinyl pyrrolidone and mixtures thereof.

4. The oral gel composition according to claim 1, wherein the gelling agent comprises a natural raw material, preferably selected from the group of celluloses and xanthans.

5. The oral gel composition according to claim 1, wherein the oral gel composition contains gelling agent in an amount of from 0.01 to 4.0% by weight, particularly preferably from 1.5 to 3.0% by weight and in particular approximately 2.0% by weight, each in relation to the total weight of the oral gel composition.

6. The oral gel composition according to claim 1, wherein the oral gel composition further comprises (c) one or more pH regulators and/or (d) one or more pharmaceutical or cosmetic ingredients.

7. The oral gel composition according to claim 6, wherein the pH regulator (d) is selected from the group consisting of acetic acid, acetates, lactic acid, lactates, malic acid, malates, fumaric acid, citric acid, citrates, tartaric acid, tartrates, orthophosphates, di-, tri- and polyphosphates, hydrochloric acid, chlorides, sulfuric acid, sulfates, hydroxides, oxides, adipic acid, adipates, gluconic acid, gluconates, calcium carbonate or comprises a hydrate thereof.

8. The oral gel composition according to claim 6, wherein the pharmaceutical or cosmetic ingredient (d) comprises one or more antimicrobial substances, flavoring agents and sugar alcohols.

9. The oral gel composition according to claim 1, wherein the oral gel composition has a pH value in the range from 5.5 to 8.5, more preferably a pH value in the range from 7.0 to 8.0, in particular a pH value of approximately 7.5.

10. The oral gel composition according to claim 1, wherein the oral gel composition is free of fluorides and/or tin salts and/or chlorhexidine and/or cetylpyridinium chloride and/or triclosan.

11. A method for treatment and/or prevention of periodontitis/gingivitis or gum recession comprising:
providing the oral gel composition according to claim 1;
using the oral gel composition for the treatment and/or prevention of periodontitis/gingivitis or gum recession.

12. The method according to claim 11, wherein the dwell time of the oral gel composition is between 3 minutes and 72 hours, preferably between 5 minutes and 48 hours, in particular between 10 minutes and 24 hours.
